(19) United States
(12) Patent Application Publication
(10) Pub. No.: US 2024/0041156 A1
(43) Pub. Date: Feb. 8, 2024

(54) ANKLE FOOT ORTHOTIC ASSEMBLY

(71) Applicants: Michael URBANOWICZ, New Vienna, OH (US); Brittany URBANOWICZ, New Vienna, OH (US)

(72) Inventors: Michael URBANOWICZ, New Vienna, OH (US); Brittany URBANOWICZ, New Vienna, OH (US)

(21) Appl. No.: 18/486,781

(22) Filed: Oct. 13, 2023

Related U.S. Application Data
(63) Continuation-in-part of application No. 17/154,102, filed on Jan. 21, 2021.

(51) Int. Cl. A43B 7/22

(52) U.S. Cl. CPC A43B 7/223

(57) ABSTRACT

An ankle foot orthotic assembly may have an ankle foot brace configured to be inserted into an interior cavity of a protective foot covering. The ankle foot brace is configured to receive a removable insole and a user's ankle and foot, the bottom portion inwardly extending along a perimeter of the rear heel portion, the internal foot side portion, the external foot side portion, the removable insole having a bottom surface configured to be inserted adjacent to a top portion of the inwardly extending bottom portion. The brace has releasable straps to secure the insole and the user's ankle and foot inside a cavity formed by the brace. The insole is configured with an angular incline from a ball of the foot regain to distal toe region at an angle of between 18 and 30 degrees.

### CROSS-REFERENCE TO RELATED APPLICATIONS

This application is a continuation-in-part of U.S. application Ser. No. 17/154,102 filed Jan. 21, 2021, entitled “Ankle Foot Orthotic Assembly”, the entire contents of which are incorporated herein by reference.

### FIELD

The disclosure relates to orthotic devices and more particularly pertains to a new orthotic device for concealing an ankle foot orthotic worn by a user. The orthotic device is integrated into a boot such that the orthotic device is concealed from an observer. Additionally, the orthotic device has an open front side to facilitate easy the orthotic device to be easily worn.

### BACKGROUND

The prior art relates to orthotic devices including a variety of boots that have an ankle foot orthotic integrated therein for treating foot drop syndrome. The prior art also discloses a variety of athletic shoes that has various means of rigid ankle supports integrated therein for enhanced ankle support of a user. The prior art additionally discloses a boot shaped cover for an ankle foot orthotic. In no instance does the prior art disclose a boot, having a flap to open the boot, in combination with an ankle foot orthotic being integrated into the boot.

### BRIEF SUMMARY OF THE INVENTION

An embodiment of the disclosure meets the needs presented above by generally comprising a boot that can be worn on a user's foot having the boot extending upwardly on the user's lower leg. The boot has a flap is integrated therein and the flap is positionable in an open position to enhance the user's ability to position their foot and lower leg into the boot. An ankle foot orthotic is integrated into the boot to engage the user's lower leg and the user's foot thereby facilitating the ankle foot orthotic to aid with foot drop syndrome in the user. Moreover, the ankle foot orthotic is positioned inside the boot thereby facilitating the ankle foot to be concealed from observers thereby enhancing the user's self-confidence.

In other embodiments an ankle foot orthotic assembly is provided that may have an ankle foot brace configured to be inserted into an interior cavity of a protective foot covering, the ankle foot brace having a rear heel portion, an internal foot side portion, an external foot side portion and a bottom portion, the ankle foot brace configured to receive a removable insole and a user's ankle and foot, the bottom portion inwardly extending along a perimeter of the rear heel portion, the internal foot side portion, the external foot side portion, the removable insole having a bottom surface configured to be inserted adjacent to a top portion of the inwardly extending bottom portion, the ankle foot brace having a plurality of releasable straps to secure the insole and the user's ankle and foot inside a cavity formed by the rear heel portion, the internal foot side portion, the external foot side portion, and bottom portion, and wherein the insole is configured with an angular incline from a ball of the foot regain to distal toe region at an angle of between 18 and 30 degrees.

In one embodiment, the ankle foot orthotic may provide a protective foot covering, such as a boot having an integral fastenable flap to allow, when opened, insertion of the ankle foot brace and to hold the ankle foot brace in place when closed and retained on the user's foot and lower leg.

Features of some of the embodiments may include each of the releasable straps is releasable by one of a hook and loop fastener and a buckle; or the fastenable flap is fastenable by one of a hook and loop fastener, a plurality of buckles or mateable zipper fasteners. Decorative laces may also be applied to a tongue portion of the boot.

The present ankle foot orthotic ankle foot brace and/or insole may be composed of at least one of a plastics, rubber, composites, graphite, fiberglass, plastics and resins in extruded form such as those sold under the tradename PORON, metal, and the like and combinations thereof.

In one approach, the releasable straps are threaded though openings in the ankle foot brace at predetermined positions.

In one approach, the insole may have a compressible resilient top portion and a rigid lower portion.

The ankle foot orthotic of present embodiments may include the boot concealing the ankle foot brace on a user when the boot is worn, the boot being configured to extend upwardly on the user's lower leg, the fastenable flap being positionable in an open position thereby exposing an interior of said boot wherein the fastenable flap and is configured to enhance the user's ability to position their foot and lower leg into the boot, the boot having mateable zipper fasteners, wherein when the ankle foot brace and insole assembly are fastened within the interior boot cavity the user's lower leg and the user's foot are engaged thereby facilitating the ankle foot orthotic to aid with foot drop syndrome in the user.

In some embodiments, the plurality of releasable straps may have three releasable straps with hook and loop fasteners, and configured so that one releasable strap is on the ankle portion of the ankle foot brace and two releasable straps are on the foot portion of the ankle foot brace.

In one approach an angular incline from a ball of the foot regain to distal toe region can be at an angle of 21 degrees.

The present embodiments may also present as a kit for an ankle foot orthotic having an ankle foot brace according to the present embodiments, an insole, and a plurality of toe inserts and/or heel inserts of varying inclines from 10 to 30 degrees.

The present embodiments may also present as a method to assemble an ankle foot orthotic having the steps of providing a kit for an ankle foot orthotic according to the present embodiments, providing a gait analysis of a user without an ankle foot orthotic, providing a gait analysis of a user with the ankle foot orthotic over a plurality of heel and/or toe insert combinations installed within a boot, selecting the heel and/or toe insert combination corresponding to the most improved gate analysis when compared against the gate analysis of a user without an ankle foot orthotic, and installing the heel and toe insert combination under the insole but within the cavity of the ankle foot brace. Additional steps may include placing the user's foot in the cavity of the cavity of the ankle foot brace, securing the user's foot within the cavity of the ankle foot brace using the plurality of releasable straps, placing the user's foot with the ankle foot brace within the boot, and securing the boot integral fastenable flap to hold the ankle foot brace in place and retained on the user's foot and lower leg.

According to other approaches of the present embodiments, the ankle foot brace can be made from a rigid TPU shell with soft EVA liners. Other embodiments may include graphite and other 3D printed composites. The ankle foot brace composition can be configured to be resilient and deformable to reduce the distance between to distal ends of the wings by up to 25 percent. The ankle foot brace and insole are in mirror image pairs configured for a user's left and right ankle and foot.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective phantom view of an ankle foot orthotic assembly according to an embodiment of the disclosure.

FIG. 2 is a back phantom view of an embodiment of the disclosure.

FIG. 3 is a front phantom view of an embodiment of the disclosure.

FIG. 4 is a perspective in-use view of an embodiment of the disclosure.

FIG. 5 is a perspective view of an exemplary right foot internal removable ankle foot orthotic brace of an embodiment of the disclosure (the left foot being a mirror image thereof).

FIG. 6 is a perspective view of an exemplary right foot internal removable ankle foot orthotic brace of an alternate embodiment of the disclosure (the left foot being a mirror image thereof).

FIG. 7 is a perspective view of an insole for a right foot ankle foot orthotic assembly (the left foot being a mirror image thereof).

FIG. 8 is an alternate perspective view of the insole according to FIG. 7 (the left foot being a mirror image thereof).

FIG. 9 is a side view of the insole according to FIG. 7 (the left foot being a mirror image thereof).

FIG. 10 is a side cross-sectional view of the insole according to FIG. 7 (the left foot being a mirror image thereof).

FIG. 11 is an exploded perspective view of a right foot ankle foot assembly using the internal removable ankle foot orthotic brace according to FIG. 5 and insole according to FIG. 7 (the left foot being a mirror image thereof).

FIG. 12 is a perspective view of the assembled right foot ankle foot orthotic assembly according to FIG. 11 with the flap open (the left foot being a mirror image thereof).

FIG. 13 is a perspective view of the assembled right foot ankle foot orthotic assembly according to FIG. 11 with the flap closed (the left foot being a mirror image thereof).

FIG. 14 is a side view of an assembled right foot ankle foot orthotic assembly according to FIG. 11 with the flap closed (the left foot being a mirror image thereof).

FIG. 15 is a side cross sectional view of an assembled right foot ankle foot orthotic assembly according to FIG. 14 with the flap closed (the left foot being a mirror image thereof).

FIG. 16 is a perspective view of an alternate exemplary right foot internal removable ankle foot orthotic brace of an embodiment of the disclosure (the left foot being a mirror image thereof).

FIG. 17 is a perspective view of an exemplary right foot internal removable ankle foot orthotic brace of an alternate embodiment of the disclosure (the left foot being a mirror image thereof).

FIG. 18 is an exploded perspective view of a right foot ankle foot assembly using the internal removable ankle foot orthotic brace according to FIG. 16 and insole according to FIG. 7 (the left foot being a mirror image thereof).

FIG. 19 is a perspective view of the assembled right foot ankle foot orthotic assembly according to FIG. 18 with the flap open (the left foot being a mirror image thereof).

FIG. 20 is a perspective view of the assembled right foot ankle foot orthotic assembly according to FIG. 18 with the flap closed (the left foot being a mirror image thereof).

FIG. 21 is a perspective view of an exemplary right foot internal removable ankle foot orthotic brace of an alternate embodiment of the disclosure (the left foot being a mirror image thereof).

FIG. 22 is a perspective view of an exemplary right foot internal removable ankle foot orthotic brace of an alternate embodiment of the disclosure (the left foot being a mirror image thereof).

FIG. 23 is a perspective view of the brace of FIG. 22 with the straps removed (the left foot being a mirror image thereof).

FIG. 24 is a top view of the exemplary right foot internal removable ankle foot orthotic brace of FIG. 22 (the left foot being a mirror image thereof).

FIG. 25 is a top view of the insole for a right foot ankle foot orthotic assembly of FIG. 7 (the left foot being a mirror image thereof).

FIG. 26 is a top view of the assembled right foot ankle foot orthotic assembly according to FIG. 13 with the flap closed (the left foot being a mirror image thereof).

FIG. 27 is a perspective view of the assembled right foot ankle foot orthotic assembly according to an alternate embodiment of the disclosure with the flap closed and decorative lacing added (the left foot being a mirror image thereof).

FIG. 28 is a side cross sectional view of an assembled right foot ankle foot orthotic assembly according to FIG. 14 with the flap closed and having a heel insert (the left foot being a mirror image thereof).

FIG. 29 is a side cross sectional view of an assembled right foot ankle foot orthotic assembly according to FIG. 14 with the flap closed and having toe and heel inserts (the left foot being a mirror image thereof).

FIG. 30 is shows a kit of an ankle foot orthotic assembly according to the present embodiments with a deformable insole and plurality of heel and toe insert sizes and optional boot (the left foot being a mirror image thereof).

### DETAILED DESCRIPTION

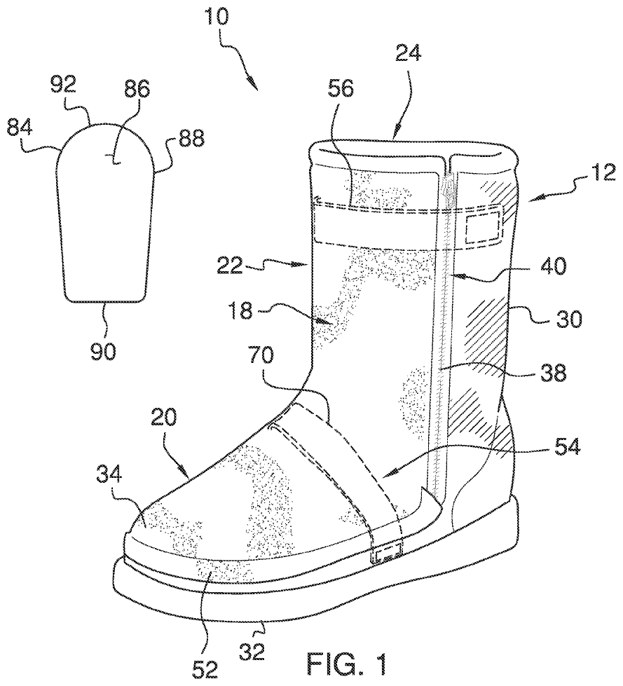

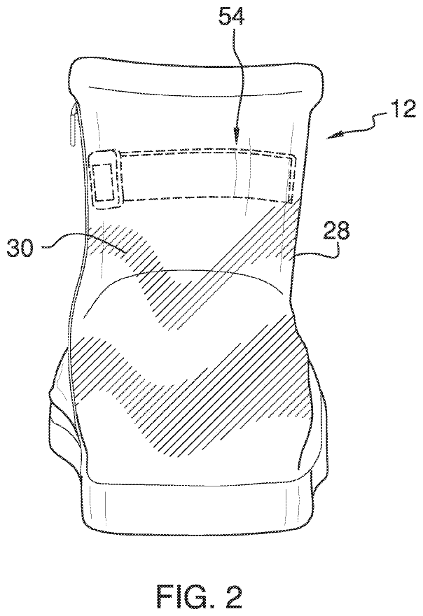

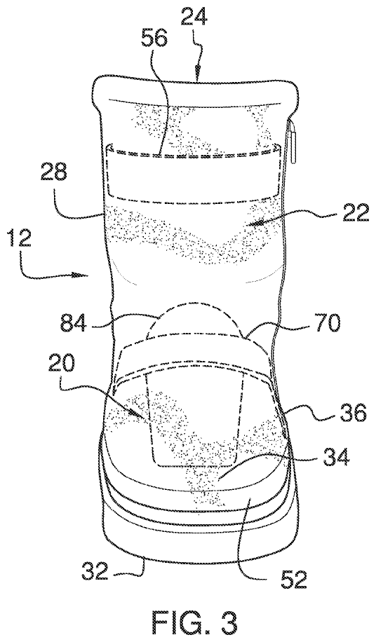

With reference now to the drawings, and in particular to FIGS. 1 through 4 thereof, a new orthotic device embodying the principles and concepts of an embodiment of the disclosure and generally designated by the reference numeral 10 will be described.

As best illustrated in FIGS. 1 through 4, the ankle foot orthotic assembly 10 generally comprises a boot 12 that can be worn on a user's foot 14 having the boot 12 extending upwardly on the user's lower leg 16. The user may be an individual with a physical disability that involves foot drop syndrome, such as a person that has suffered a stroke, a person that suffers from cerebral palsy or any other type of disability that involves foot drop syndrome. The boot 12 has a flap 18 integrated therein and the flap 18 is positionable in an open position thereby exposing an interior of the boot 12. In this way the flap 18 enhances the user's ability to position their foot 14 and lower leg 16 into the boot 12. Conversely, the flap 18 is positionable in a closed position retain the boot 12 on the user's foot 14 and lower leg 16.

The boot 12 has a foot portion 20 that surrounds the user's foot 14 and the boot 12 has a leg portion 22 extending upwardly from the foot portion 20 to surround the user's lower leg 16. The leg portion 22 has a top end 24 that is open to define an opening 26 into the leg portion 22 to facilitate the user's lower leg 16 to extend therethrough. The leg portion 22 has an outward side 28 that is positioned on a lateral aspect 29 of the user's lower leg 16 when the boot 12 is worn. Additionally, the leg portion 22 has a back quarter 30 and the back quarter 30 is comprised of a rigid material to assist with supporting the user's lower leg 16 and the user's foot 14 when the boot 12 is worn. The boot 12 may be comprised of leather or any other type of material that meets the user's preference for the overall style and appearance of the boot 12.

The foot portion 20 has a sole 32 and a toe 34, and the foot portion 20 has an inward side 36 that is positioned on a medial side 37 of the user's foot 14 when the boot 12 is worn. The boot 12 has a cut 38 extending therethrough, and the cut 38 has a first portion 40 extending downwardly from the top end 24 of the leg portion 22 along the outward side 28 of the leg portion 22. Additionally, the cut 38 has a second portion 42 extending along the foot portion 20 of the boot 12. The second portion 42 is spaced from the sole 32 and the second portion 42 is oriented to extend along a line that is oriented perpendicular to the first portion 40. Moreover, the second portion 42 extends around the toe 34 to terminate on the inward side 36 of the foot portion 20. In this way the first portion 40 and the second portion 42 of the cut 38 defines the flap 18, and the cut 38 has a first edge 44 and a second edge 46.

A first fastener 48 is coupled to the boot 12 and the first fastener 48 is coextensive with the first edge 44 of the cut 38. A second fastener 50 is coupled to the flap 18 and the second fastener 50 is mateable to the first fastener 48 for retaining the flap 18 in the closed position. The second fastener 50 is un-mateable from the first fastener 48 to facilitate the flap 18 to be positioned in the open position, and the second fastener 50 is coextensive with the second edge 46 of the cut 38. Each of the first fastener 48 and the second fastener 50 may comprise complementary halves of a zipper or other type of mechanically releasable fastening device.

A cover 52 is provided and the cover 52 is coupled to the boot 12. The cover 52 covers a substantial amount of the first fastener 48 and the second fastener 50 when the flap 18 is in the closed position. In this way the cover 52 inhibits an observer from seeing the first fastener 48 and the second fastener 50 thereby enhancing the user's self-confidence the user is wearing the boot 12. The cover 52 is positioned on the foot portion 20 of the boot 12 and the cover 52 is coextensive with the second portion 42 of the cut 38. Moreover, the cover 52 extends downwardly over the second portion 42 of the cut 38 when the flap 18 is in the closed position for concealing the first fastener 48 and the second fastener 50 along the foot portion 20 of the boot 12. The boot 12 may be manufactured to custom specifications as required by orthotic specialists to facilitate a custom fit for any user.

An ankle foot orthotic 54 is integrated into the boot 12 and the ankle foot orthotic 54 engages the user's lower leg 16 and the user's foot 14 when the user wears the boot 12. In this way the ankle foot orthotic 54 can aid with foot drop syndrome in the user.

The ankle foot orthotic 54 is positioned inside the boot 12 such that the ankle foot orthotic 54 is concealed from observers thereby enhancing the user's self-confidence.

The ankle foot orthotic 54 comprises a first strap 56 that has a first end 58, a second end 60 and a first surface 62 extending therebetween, and the first strap 56 is elongated between the first end 58 and the second end 60. The first end 58 is coupled to an inside surface 64 of the back quarter 30 of the leg portion 22 of the boot 12 at a point that is located on an opposite side of the leg portion 22 with respect to the cut 38. Moreover, the first strap 56 is oriented to extend along a horizontal axis thereby facilitating the first strap 56 to be wrapped around the user's lower leg 16 when the user wears the boot 12. The first strap 56 is spaced from the top end 24 of the leg portion 22.

The ankle foot orthotic 54 includes a first mating member 66 that is coupled to the first surface 62 of the first strap 56. The first mating member 66 is positioned adjacent to the second end 60 of the first strap 56. Additionally, the ankle foot orthotic 54 includes a second mating member 68 that is coupled to the inside surface 64 of the back quarter 30 of the leg portion 22 of the boot 12 at a point is located adjacent to the first edge 44 of the cut 38. The second mating member 68 is positioned adjacent to the top end 24 of the leg portion 22 and the first mating member 66 is releasably mateable to the second mating member 68. Each of the first mating member 66 and the second mating member 68 may comprise a hook and loop fastener or other type of releasable fastener.

The ankle foot orthotic 54 includes a second strap 70 that has a primary end 72, a secondary end 74 and a primary surface 76 extending therebetween, and the second strap 70 is elongated between the primary end 72 and the secondary end 74. The primary end 72 is coupled to an inside surface 78 of the foot portion 20 of the boot 12 at a point that is located on the same side of the boot 12 with respect to the first strap 56. Additionally, the second strap 70 is centrally positioned between the leg portion 22 of the boot 12 and the toe 34 of the foot portion 20 of the boot 12. In this way the second strap 70 can extend over the 79 instep of the user's foot 14 when the user wears the boot 12.

The ankle foot orthotic 54 includes a primary mating member 80 that is coupled to the primary surface 76 of the second strap 70. The primary mating member 80 is positioned adjacent to the secondary end 74 of the second strap 70. The ankle foot orthotic 54 includes a secondary mating member 82 that is coupled to the inside surface 64 of the foot portion 20 of the boot 12 at a point that is located adjacent to the first edge 44 of the cut 38. The secondary mating member 82 is centrally positioned between the leg portion 22 of the boot 12 and the toe 34 of the foot portion 20 of the boot 12. Additionally, the primary mating member 80 is releasably mateable to the secondary mating member 82. Each of the primary mating member 80 and the secondary mating member 82 may comprise a hook and loop fastener or other type of releasable fastener.

An instep pad 84 that is positionable beneath the second strap 70 when the ankle foot orthotic 54 is worn to enhance comfort for the user's foot 14. The instep pad 84 has a first surface 86 and a perimeter edge 88, and the first surface 86 of the instep pad 84 is concavely arcuate with respect to the perimeter edge 88. In this way the first surface 86 can conform to curvature of the instep 79 of the user's foot 14. The perimeter edge 88 has a front side 90 and a back side 92, the back side 92 extends along a straight line and the front side 92 is rounded. Additionally, the instep pad 84 is comprised of a resiliently compressible material.

In use, the flap 18 is positioned in the open position to facilitate the user to wear the boot 12. The first strap 56 is extended across the user's lower leg 16 to facilitate the first mating member 66 to be mated to the second mating member 68. The second strap 70 is extended over the user's foot 14 to facilitate the primary mating member 80 to be mated to the secondary mating member 82. The flap 18 is positioned in the closed position and the first fastener 48 and second fastener 50 are mated to close the flap 18. In this way the boot 12 facilitates the ankle foot orthotic 54 to be employed by the user in a stylish manner that is visually appealing and that additionally conceals the ankle foot orthotic 54 from observers.

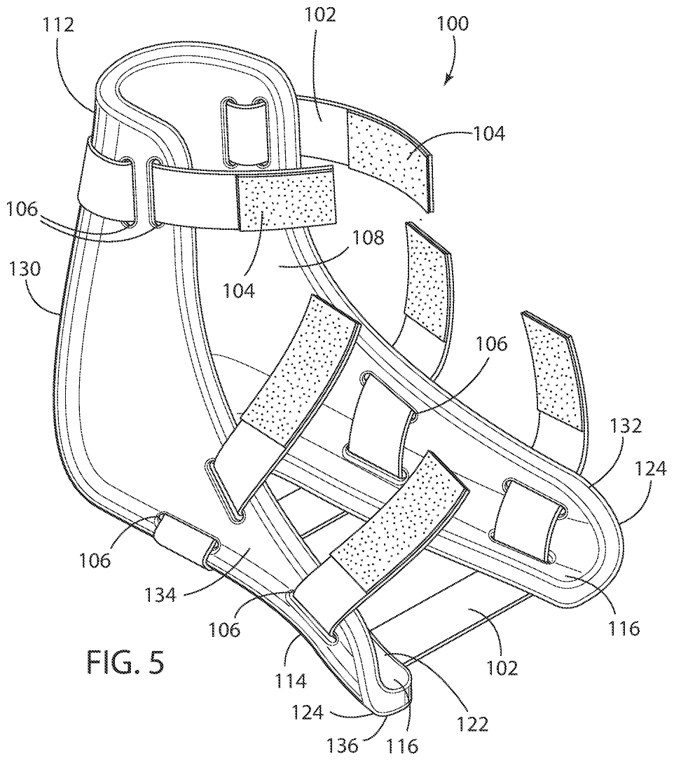

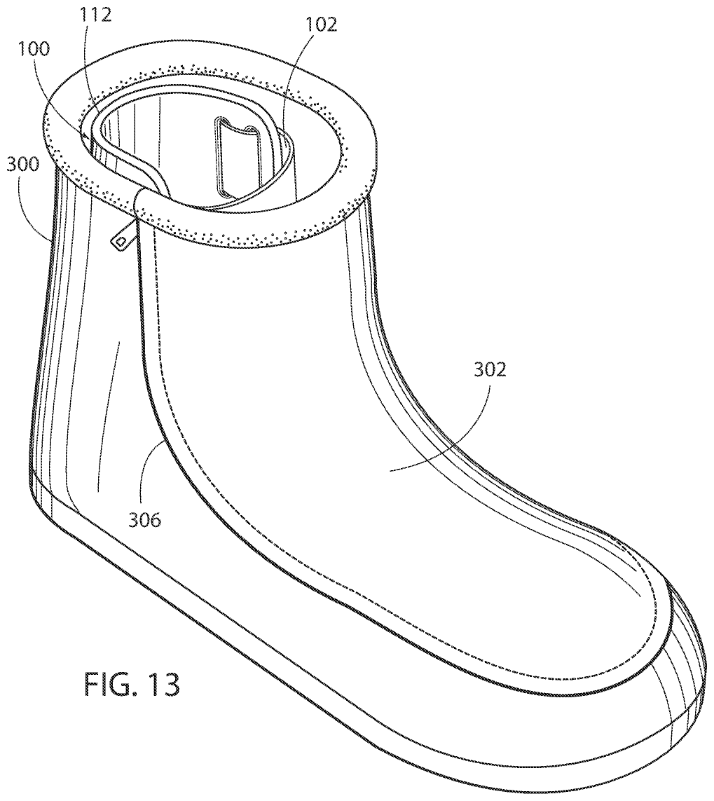

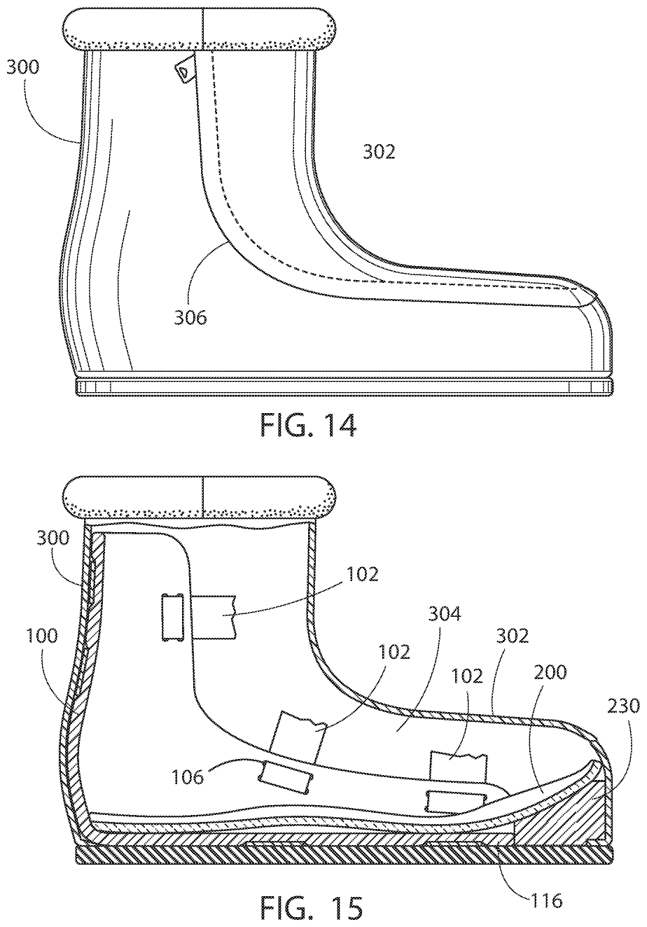

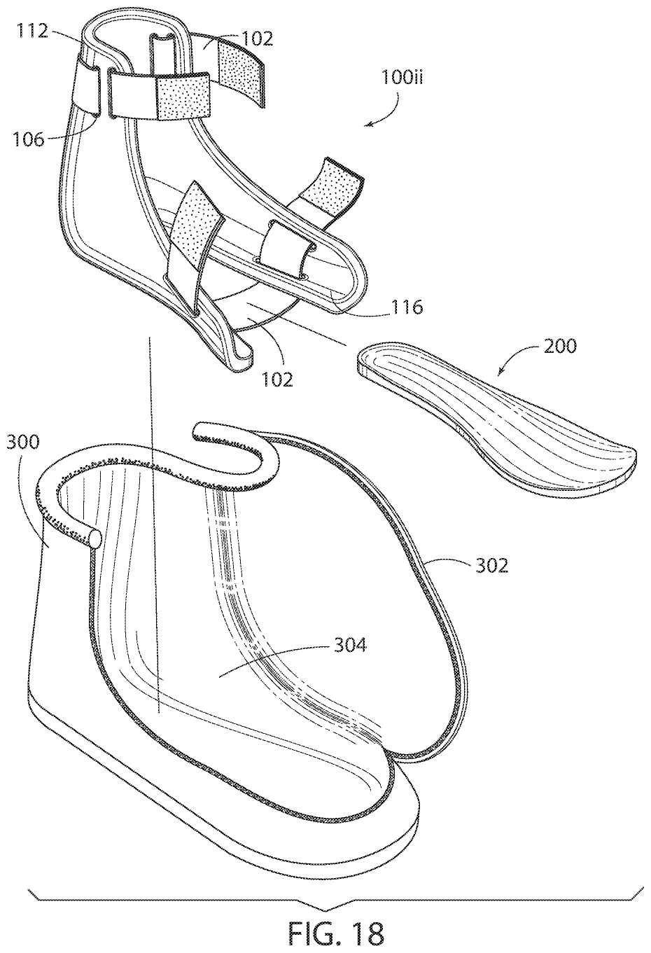

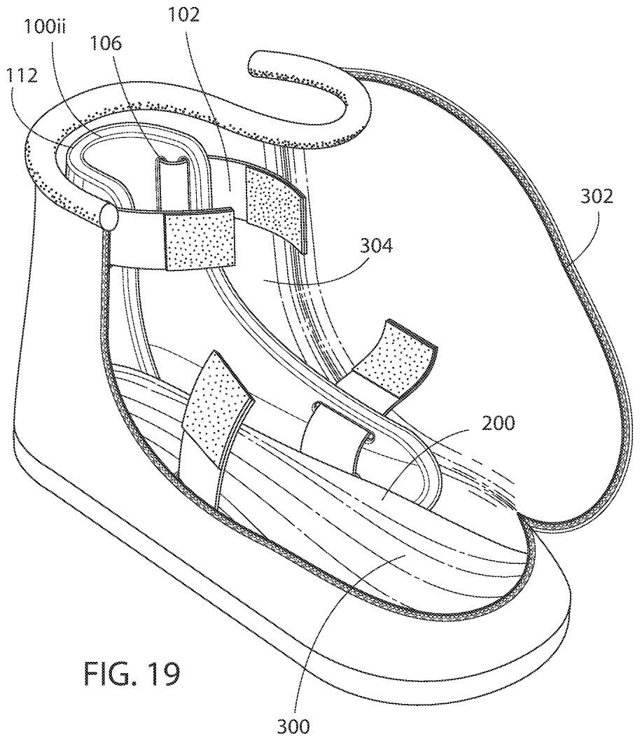

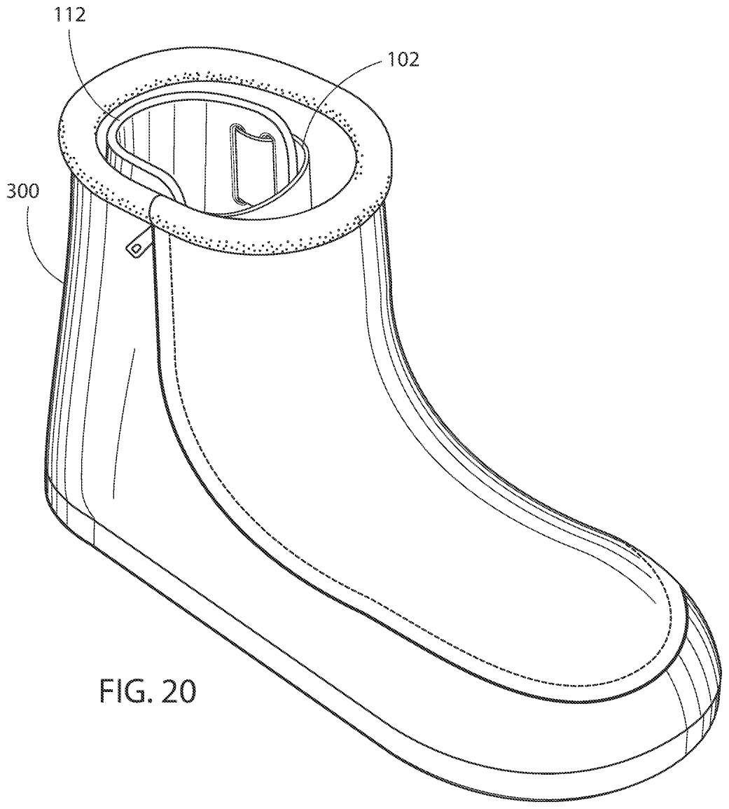

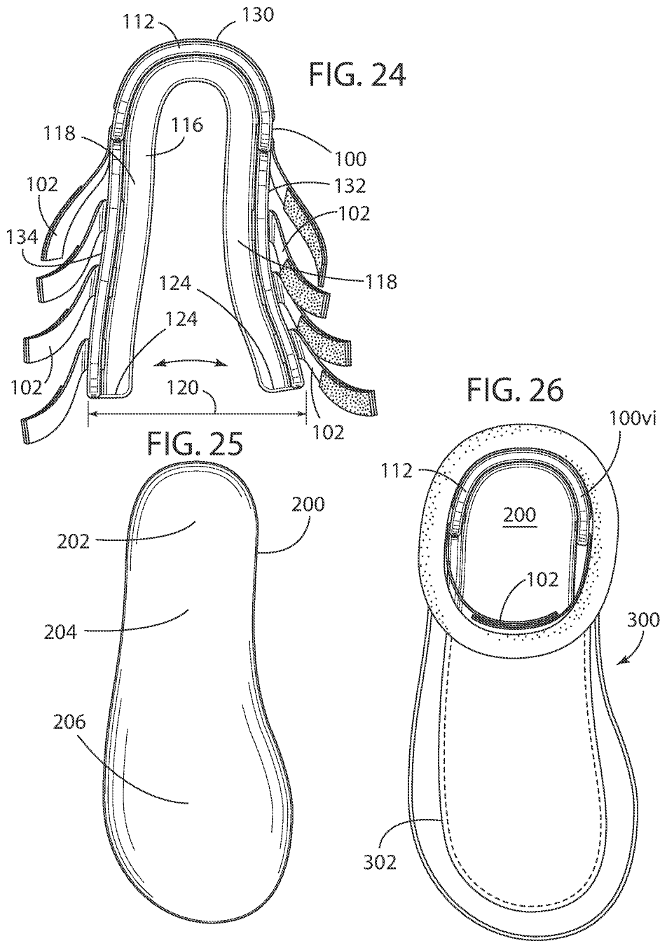

The present embodiments represent an improvement in orthotic technology, particularly ankle foot orthoses (AFOs) and/or supramalleolar orthoses (SMOs) in key areas of a user's gait. The results provide freedom, movement, and quality of life for a user. The utility of the present embodiments is clear in its performance compared to prior art AFO's. In direct comparisons, the tables below demonstrate the capability of the present embodiments to enhance walking speed by 14.86% and cadence by 5.93% compared to traditional AFOs. Such improvements translate to improved mobility, increased independence, and considerable enhancement in the user's quality of life. In a comprehensive gait lab analysis of the embodiments shown in FIGS. 5-27, the present embodiments exhibited superior performance, overall outperforming the prior AFOs by 5.1%. See tables below:

Performance Evaluation
Comparing the ankle foot orthotic to traditional Ankle Foot Orthoses
(AFOs) across six key gait parameters: Speed, Cadence,
Stance, Swing, Step/Leg Length Ratio, and Initial Double Support.
Results:

| | Present ankle foot | Prior art | Percentage |
|---|---|---|---|
| Gait Parameter | orthotic embodiment | AFO | Difference |
| Speed | 0.85 | 0.74 | +14.86% |
| Cadence | 125 | 118 | +5.93% |

As for the Stance, Swing, Step/Leg Length Ratio, and Initial Double
Support parameters, both the present embodiment
and the prior art AFO scored zero, resulting in a 0% difference.

Differences in gait parameters between the left and
right feet were also examined.

| | | Prior art | Percentage |
|---|---|---|---|
| Gait Parameter | Left present embodiment | LAFO | Difference |
| Stance | 62 | 64 | −3.13% |
| Swing | 38 | 36 | +5.56% |
| Step/Leg Length | 78 | 73 | +6.85% |
| Ratio | | | |
| Initial Double | 11 | 14 | −21.43% |
| Support | | | |
| | | | Percentage |
| Gait Parameter | Right present embodiment | RAFO | Difference |
| Stance | 56 | 57 | −1.75% |
| Swing | 44 | 43 | +2.33% |
| Step/Leg Length | 87 | 79 | +10.13% |
| Ratio | | | |
| Initial Double | 7 | 8 | −12.5% |
| Support | | | |

The present embodiments deliver balanced performance across both of a user's legs, offering excellent adaptability. Seamlessly integrating the present AFO embodiments within a regular piece of footwear, for example a boot, improves the medical device aesthetic, reducing the stigma often associated with orthotic use. This innovative approach fosters a greater sense of comfort and confidence among users, significantly improving their quality of life.

The present embodiments may be realized in a variety of compositions such as: Carbon Fiber AFOs, Plastic AFOs, Metal AFOs, and the like and combinations thereof. Carbon Fiber is known for its strength and lightweight properties, while Plastic embodiments may be more affordable but may not offer the same level of durability and performance. Metal embodiments may also be considered. Specifically, the present embodiments, particularly its insole and ankle brace portions, can be constructed from various materials such as plastics, rubber, composites, graphite, fiberglass, plastics and resins in extruded form such as those sold under the tradename PORON, metal, and the like and combinations thereof. Each material has unique properties, and the aim is to balance durability, flexibility, and user comfort. In a preferred embodiment, a combination of a rigid thermoplastic polyurethane (TPU) shell with soft ethylene-vinyl acetate (EVA) liners may be used. Other embodiments may include graphite and other 3D printed composites.

The flexibility of the present embodiments, especially in the toe portion, is designed to allow for a natural uplift in the toe portion. The inclined effect, either by a rigid insole or by a predetermined toe insert is comparable to walking in high heels without the hill portion on the back end of the foot. This toe incline is to naturally shift the body's weight to a more natural gait form with a user with cerebral palsy.

While the present embodiments provide a degree of deformability to mold to the user's foot shape, it also provides some elasticity and resiliency to ensure ensures the brace returns to its original shape after being stretched or compressed, thus maintaining its structural integrity over time.

With reference now to the drawings, and in particular to FIGS. 5-30 thereof, new orthotic devices embodying the principles and concepts of ankle foot orthotic assembly embodiments of the disclosure are shown.

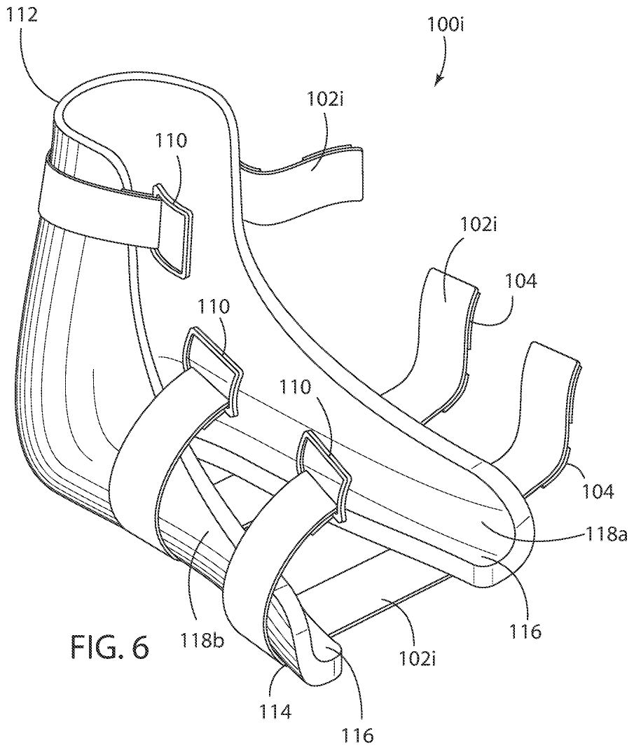

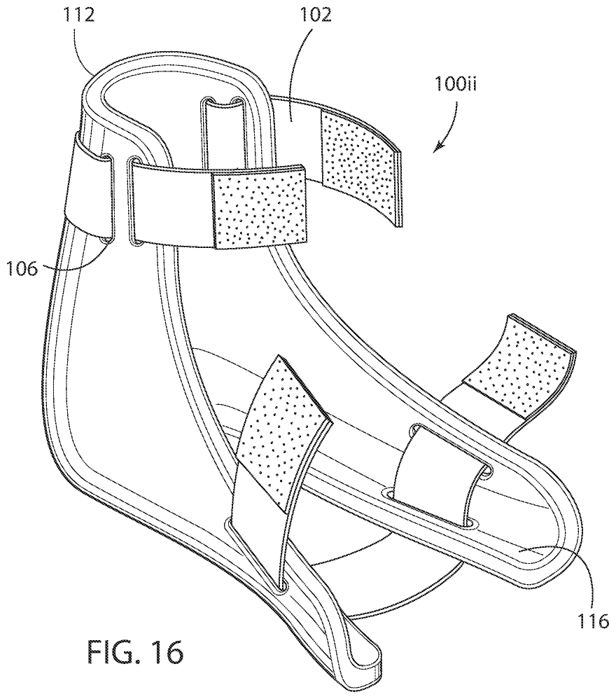

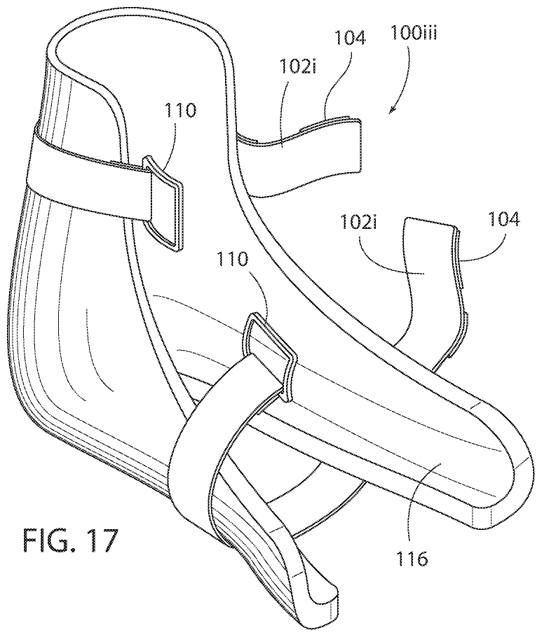

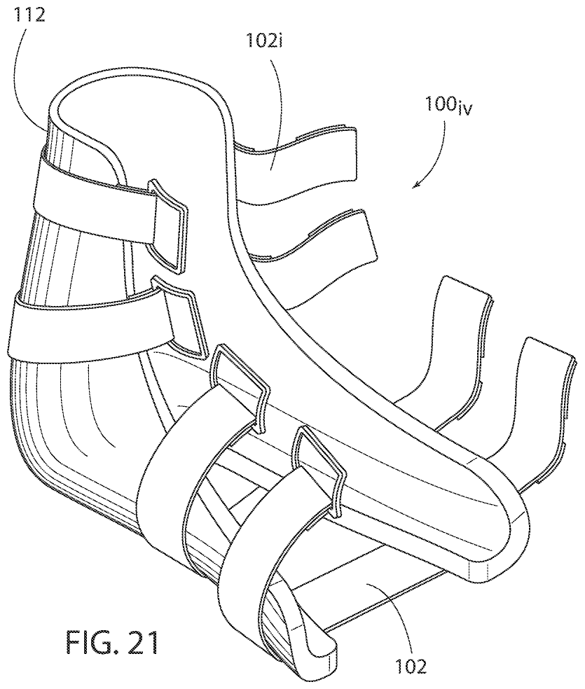

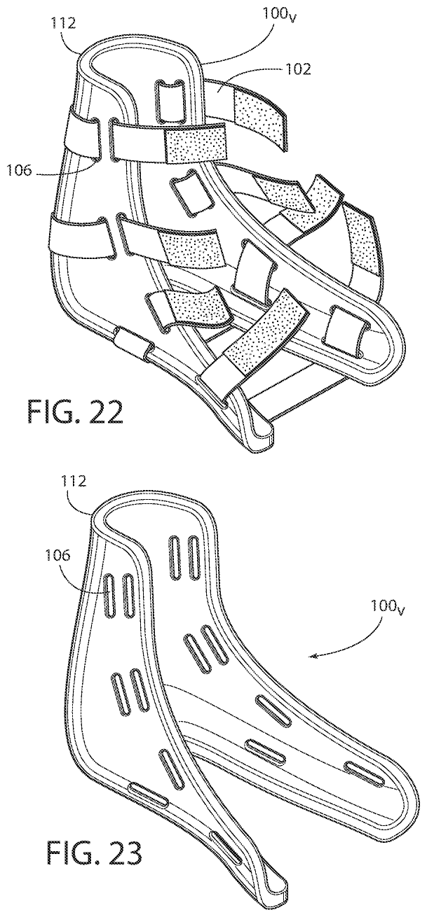

FIG. 5 shows internal orthotic brace 100 according to one approach (the insole 200 is not shown but would be present when assembled and in use) and has three hook and loop straps 102. Alternate internal orthotic brace 100i may be found in FIG. 6 and has three optionally buckled hook and loop straps 102i. Alternate internal orthotic brace 100ii may be found in FIG. 16 and has two hook and loop straps 102. Alternate internal orthotic brace 100iii may be found in FIG. 17 and has two buckled hook and loop straps 102i. Alternate internal orthotic brace 100iv may be found in FIG. 21, and has four buckled hook and loop straps 102i. Alternate internal orthotic brace 100v may be found in FIG. 22 and has four hook and loop straps 102. The hook and loop fasteners 104 used may be those such as sold under the tradename VELCRO. As shown in FIG. 23, the internal orthotic brace 100 may have predetermined brace openings 106 sized to receive straps 102. In some embodiments many openings 106 may be provided to allow for various combinations and numbers of straps 102

Orthotic device 100 has an interior cavity 108 with an ankle portion 112, a foot portion 114, an internal orthotic brace inwardly extending edge 116, and forward extending ‘wings’ 118. As shown in FIG. 25, a distance 120 spanning between the opening of a distal end 124 of the forward extending ‘wings’ 118. This allows for some deformation to friction fit a portion of insole 200 adjacent to a top portion 122 of the brace inwardly extending edge 116. The deformation can also allow for securing the user's ankle and foot when the straps 102 are secured. The remaining portion of insole 200 extends beyond the orthotic brace 100 such as shown in FIG. 15. As shown in FIG. 5, brace 100 has a rear heel portion 130, internal/medial/inner foot side portion 132, external/outer foot side portion 134, and bottom portion 136.

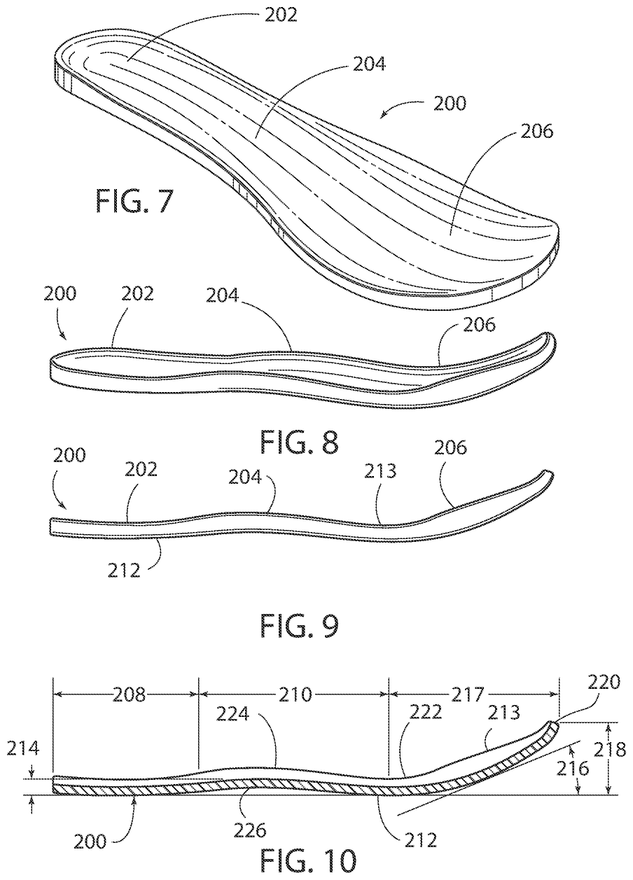

FIGS. 7-10 show an exemplary insole 200, which depending on the configuration may be rigid, resilient and deformable and composed of plastics, rubber, composites, graphite, fiberglass, closed cell foam, gel, resins, plastics and resins in extruded form such as those sold under the tradename PORON, metal, and the like and combinations thereof. Insole 200 of ankle foot orthotic device 100 has an exemplary insole portion 200, heel portion 202, mid-sole portion 204, bottom surface 212, top surface 213, and fore-foot portion 206.

As shown in FIG. 10, a heel portion 202 of insole 200 may be contoured at 208 to the shape of the user's heel. Mid-sole portion 204 can be contoured to a user's instep/arch 210 heel dimension 214. And, fore-foot portion 206 can be contoured to the ball-of-foot and toe portion 217. Within the ball-of-foot and toe portion 217 region an angular incline 216 of about 18-30 degrees may be provided at the ball-of-foot to toe portion of FIG. 10 relative to the plane of insole 200 portion and can raise, for example, to an insole 200 height 218 at tip 220 compared to bottom surface 212 of insole 200. The incline should be configured to start at 222, at the metatarsophalangeal joint (MCP) at the base of a user's toe.

Insole 200 may have an insole top layer 224 and an insole bottom layer 224. Top layer 224 may be a gel or foam to provide comfort to a user, while the bottom layer 224 may provide support and rigidity to provide the desired toe inclination and be formed of a rigid plastic or composite and the like.

As shown in FIGS. 7-10, 15, 28 and 29, the present insole embodiments may be configured with an angular incline from a ball of the foot regain to distal toe region at an angle of between 18 and 30 degrees. In one embodiment the incline starts at about 60 mm back from tip 220 of the insole and is raised about 25 mm above the bottom surface 212 of insole 200 rearward from the incline. The degree and pitch of the toe insert angle may need to be adjusted depending on the individual for optimal improved gait.

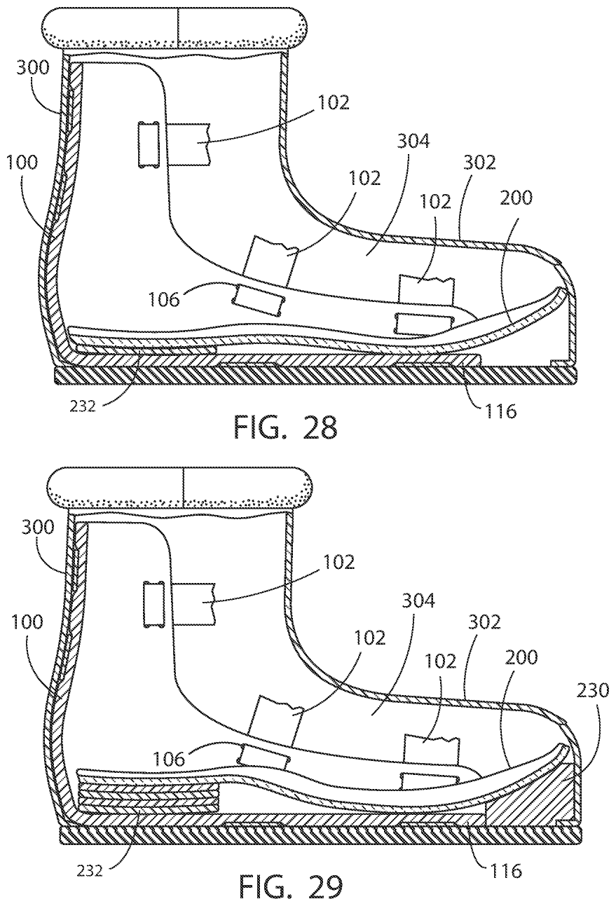

Optionally, in some embodiments a flexible insole 200, such as a gel insole, may be provided and raised and/or inclined by the insertion of rigid/semi-rigid inserts. As shown in FIGS. 28 and 29, toe insert 230 and insole heel insert 232 are provided. Heel insert 232 may provide an elevation of about 5 mm to 30 mm. Several heel inserts 232 can be provided and stacked as needed. Toe insert 230 may provide, for example, an inclined elevation of about 2 mm rising to 5 mm to 30 mm in height. In one embodiment a 10 mm heel insert may be added. In another embodiment, a toe insert may rise to a height 218 of 25 mm.

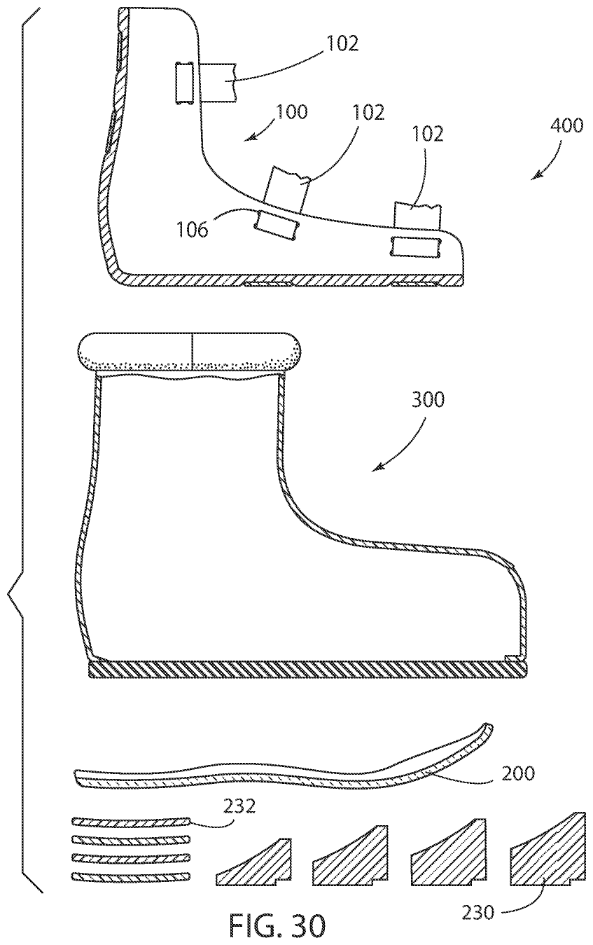

In some embodiments, a kit 400 may be provided having an ankle foot orthotic with straps, an insole and a plurality of various sizes and shapes of toe inserts 230 and heel inserts 232 (See, FIG. 30). A user may perform a gait analysis to determine the best configuration for optimized gait.

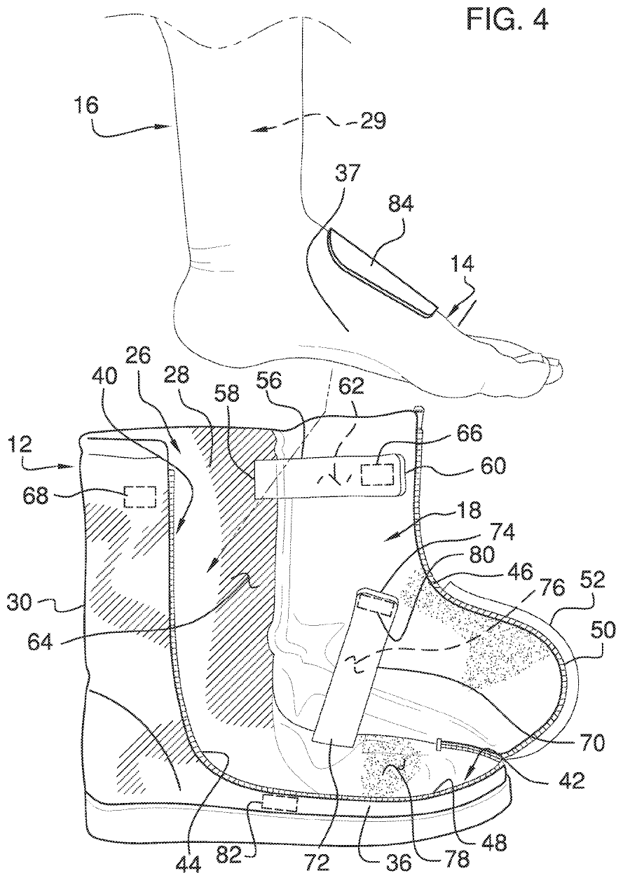

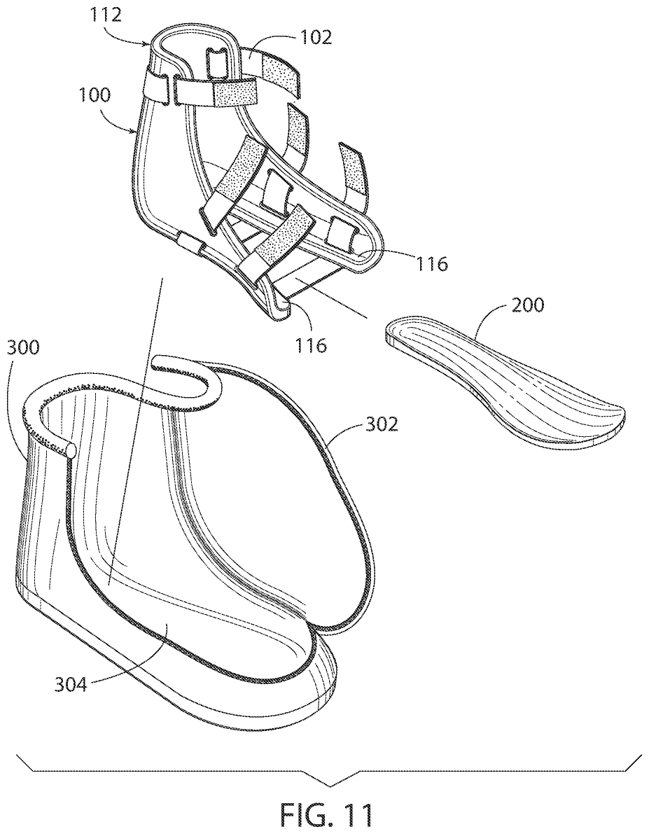

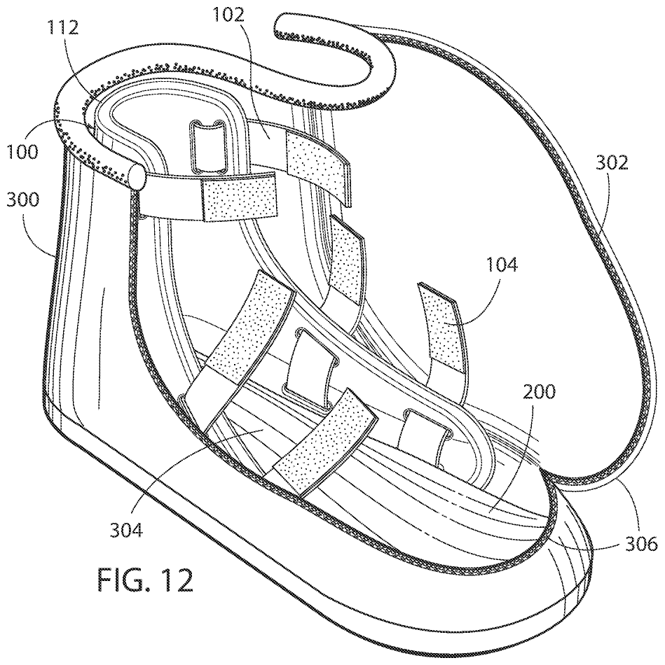

Optional footwear may also be provided. For example, FIG. 12, shows exemplary boot 300 configured to receive the internal orthotic device/insole assembly of the present embodiments. As shown, boot 300 has a boot flap 302 and an interior cavity 304 to receive the internal orthotic device/insole assembly. A flap fastener 306 (e.g., zipper, hook and loop fastener and the like) may be provided to hold the user's ankle, foot, and internal orthotic brace in place while ambulating. FIGS. 11 and 12 show for example, a boot flap fastener similar to fasteners 48 and 50 in boot 12 of FIG. 4.

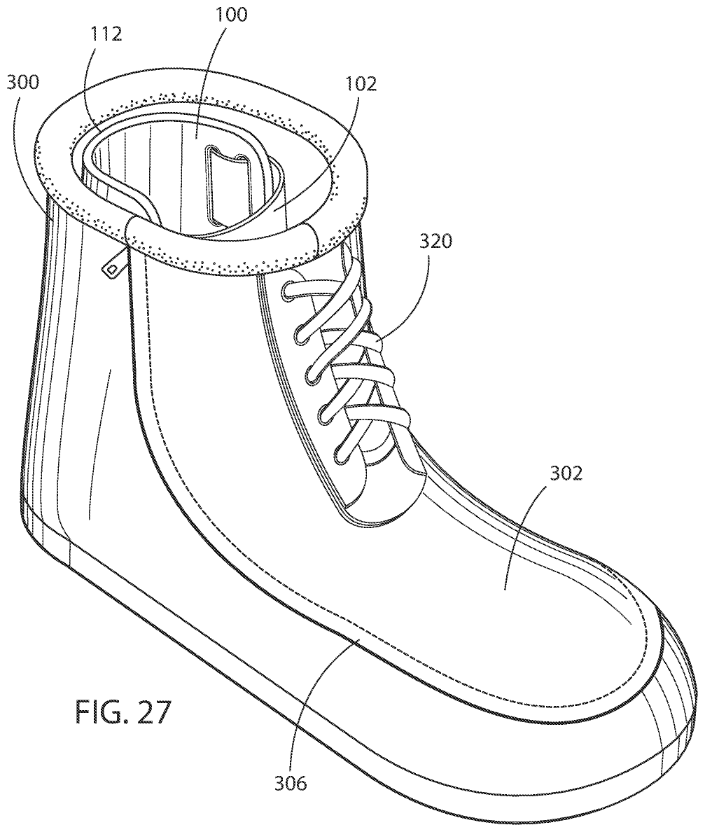

Further improvements to the boot may include aesthetically pleasing features such as laces, light emitting diodes, or other shoe decorations. By way of a non-limiting example, FIG. 27 shows an embodiment have decorative laces 320 applied to the fastenable flap tongue portion of the boot.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements. Throughout this specification numerical labels of previously shown or discussed features may be reused to indicate similar features.

1-16. (canceled)

17. An ankle foot orthotic assembly, comprising:
an ankle foot brace configured to be inserted into an interior cavity of a protective foot covering,
the ankle foot brace having a rear heel portion, an internal foot side portion, an external foot side portion and a bottom portion,
the ankle foot brace configured to receive a removable insole and a user's ankle and foot, the bottom portion inwardly extending along a perimeter of the rear heel portion, the internal foot side portion, the external foot side portion,
the removable insole having a bottom surface configured to be inserted adjacent to a top portion of the inwardly extending bottom portion,
the ankle foot brace having a plurality of releasable straps to secure the insole and the user's ankle and foot inside a cavity formed by the rear heel portion, the internal foot side portion, the external foot side portion, and bottom portion, and
wherein the insole is configured with an angular incline from a ball of the foot regain to distal toe region at an angle of between 18 and 30 degrees.

18. The ankle foot orthotic of claim 17, wherein the protective foot covering is a boot having an integral fastenable flap to allow, when opened, insertion of the ankle foot brace and to hold the ankle foot brace in place when closed and retained on the user's foot and lower leg.

19. The ankle foot orthotic of claim 17, wherein each of the releasable straps is releasable by one of a hook and loop fastener and a buckle.

20. The ankle foot orthotic of claim 18, wherein the fastenable flap is fastenable by one of a hook and loop fastener and a buckle.

21. The ankle foot orthotic of claim 17, ankle foot brace and/or insole are composed of at least one of a plastics, rubber, composites, graphite, fiberglass, plastics and resins in extruded form such as those sold under the tradename PORON, metal, and the like and combinations thereof.

22. The ankle foot orthotic of claim 17, wherein the releasable straps are threaded though openings in the ankle foot brace at predetermined positions.

23. The ankle foot orthotic of claim 17, wherein the insole is comprised of a compressible resilient top portion and a rigid lower portion.

24. The ankle foot orthotic of claim 18, wherein the decorative laces are applied to a tongue portion of the boot.

24. The ankle foot orthotic of claim 18, wherein the boot conceals the ankle foot brace on a user when the boot is worn,
the boot being configured to extend upwardly on the user's lower leg,
the fastenable flap being positionable in an open position thereby exposing an interior of said boot wherein the fastenable flap and is configured to enhance the user's ability to position their foot and lower leg into the boot,
the boot having mateable zipper fasteners,
wherein when the ankle foot brace and insole assembly are fastened within the interior boot cavity the user's lower leg and the user's foot are engaged thereby facilitating the ankle foot orthotic to aid with foot drop syndrome in the user.

25. The ankle foot orthotic of claim 17, wherein the plurality of releasable straps comprises three releasable straps with hook and loop fasteners, and configured so that one releasable strap is on the ankle portion of the ankle foot brace and two releasable straps are on the foot portion of the ankle foot brace.

26. The ankle foot orthotic of claim 17, wherein an angular incline from a ball of the foot regain to distal toe region at an angle of 21 degrees.

27. A kit for an ankle foot orthotic, comprising:
an ankle foot brace according to claim 18, an insole, and
a plurality of toe inserts and/or heel inserts of varying inclines from 10 to 30 degrees.

28. A method to assemble an ankle foot orthotic, comprising:
providing a kit for an ankle foot orthotic according to claim 27,
providing a gait analysis of a user without an ankle foot orthotic,
providing a gait analysis of a user with the ankle foot orthotic over a plurality of heel and/or toe insert combinations installed within a boot,
selecting the heel and/or toe insert combination corresponding to the most improved gate analysis when compared against the gate analysis of a user without an ankle foot orthotic, and
installing the heel and toe insert combination under the insole but within the cavity of the ankle foot brace.

29. The method of claim 28, further comprising,
placing the user's foot in the cavity of the cavity of the ankle foot brace, securing the user's foot within the cavity of the ankle foot brace using the plurality of releasable straps,
placing the user's foot with the ankle foot brace within the boot, and
securing the boot integral fastenable flap to hold the ankle foot brace in place and retained on the user's foot and lower leg.

30. The ankle foot orthotic of claim 17, wherein the ankle foot brace is composed of a rigid TPU shell with soft EVA liners. Other embodiments may include graphite and other 3D printed composites.

31. The ankle foot orthotic of claim 30, wherein the ankle foot brace composition is configured to be resilient and deformable to reduce the distance between to distal ends of the wings by up to 25 percent.

32. The ankle foot orthotic of claim 17, wherein the ankle foot brace and insole are in mirror image pairs configured for a user's left and right ankle and foot.
